(19) United States
(12) Patent Application Publication
(10) Pub. No.: US 2018/0002483 A1
(43) Pub. Date: Jan. 4, 2018

(54) BIOACTIVE COMPOSITES WITH FUNCTION OF RADIOPACITY

(71) Applicant: MontJade Engineering Co., Ltd., Taichung City (TW)

(72) Inventors: JIUNN-LIANG CHEN, Taichung (TW); JEN-CHANG YANG, Taichung (TW)

(73) Assignee: MontJade Engineering Co., Ltd.

(21) Appl. No.: 15/638,873

(22) Filed: Jun. 30, 2017

(30) Foreign Application Priority Data
Jun. 30, 2016 (TW) 105120679

(51) Int. Cl. C08G 65/40; A61L 27/54; A61L 27/04; C08K 3/08; C08K 3/22; A61L 27/56; C08K 3/30

(52) U.S. Cl. CPC C08G 65/4012; C08K 3/22; A61L 27/54; A61L 27/56; A61L 27/04; C08K 3/08; C08K 2003/2206; C08K 2003/2275; C08K 2003/3045

(57) ABSTRACT

A bioactive composite includes 10% to 40% by weight of calcium sulfate (CaSO₄), 10% to 20% by weight of tantalum pentoxide (Ta₂O₅), and 40% to 80% of polyetheretherketone (PEEK). Calcium sulfate is anhydrous calcium made by removing crystallization water of beta calcium sulfate hemihydrate.

### BACKGROUND OF THE INVENTION

### 1. Technical Field

The present invention relates to a bioactive composite, and more particularly to a bioactive composite with function of radiopacity for surgery.

### 2. Description of Related Art

As high development of theory and technique of manufacturing and biomaterial usage in recent years, titanium and titanium alloys are replaced by high-performance biopolymers in surgical implants. For a variety of biopolymer materials today, polyetheretherketone (PEEK) has an elastic modulus relatively close to that of human bones, so that when it is implanted, it could reduce bone resorption and bone atrophy caused by stress shielding in biomechanics. Furthermore, PEEK has the functions of chemical resistance, fatigue resistance, stable physicochemical property to take sterilization operation by autoclaving, ethylene oxide, or gamma radiation, so that PEEK is a good long term implant (Biomaterials 2007, 28:4845-4869). However, PEEK has a weak link with human bones due to its low bioactivity.

According to biomimetics of bone bioengineering, pore size and porosity of the implants affect cell adhesion and bone ingrowth. The interconnected pores benefit ingrowth of blood vessels and metabolism. However, porous scaffolds usually reveal the following properties: inorganic porous scaffolds are brittle and biopolymer porous scaffolds have weak strength. Therefore, porous bone implanting scaffolds are insufficient to provide the trauma tissues a stable mechanical environment when they are used for the treatment of degenerative disease of lumbar spine or cervical spine.

Another problems of PEEK are its lack of bioactive and radio translucency which makes it can't be shown in X-ray film. All these problems should be fixed.

### BRIEF SUMMARY OF THE INVENTION

In view of the above, the primary objective of the present invention is to provide a bioactive composite with functions of radiopacity and in site pore forming after implanting in tissues.

In order to achieve the objective of the present invention, a bioactive composite includes 10% to 40% by weight of calcium sulfate (CaSO₄), 10% to 20% by weight of tantalum pentoxide (Ta₂O₅), and 40% to 80% of polyetheretherketone (PEEK). Anhydrous calcium sulfate is made by removing moisture from beta calcium sulfate hemihydrate.

In an embodiment, the further bioactive composite includes up to 10% by weight of barium sulfate (BaSO₄).

In an embodiment, the further bioactive composite includes up to 10% by weight of ferrous ferric oxide (Fe₃O₄).

The bioactive composite of the present invention has the function of radiopacity, and overcoming the problems of the conventional PEEK implants.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a flowchart of the manufacturing and testing processes of a preferred embodiment of the present invention;

FIG. 2A is a diagram of the preferred embodiment of the present invention, showing the sizes of the bioactive composites (calcium sulfate);

FIG. 2B is sketch diagram of the preferred embodiment of the present invention, showing how the bioactive composite forming the pores after being implanted;

FIG. 3 is a sketch diagram of the preferred embodiment of the present invention, showing the reference gray scale relating to the thicknesses of aluminum corresponding to the X-ray radiopacity;

FIG. 4 is a sketch diagram of the preferred embodiment of the present invention, showing the X-ray radiopacity of ten composites with different compositions;

FIG. 5A is a sketch diagram of the preferred embodiment of the present invention, showing the melting temperatures (Tₘ) of the ten composites with different compositions;

FIG. 5B is a sketch diagram of the preferred embodiment of the present invention, showing the crystallization temperatures (T_{c}) of the ten composites with different compositions;

FIG. 5C is a sketch diagram of the preferred embodiment of the present invention, showing the measurement results of the DSC (differential scanning calorimeter);

FIG. 6 is a sketch diagram of the preferred embodiment of the present invention, showing the TGA (thermogravimetric analysis) of the ten composites with different compositions; and

FIG. 7 is a sketch diagram of the preferred embodiment of the present invention, showing the biological compatibility of the ten composites with different compositions.

### DETAILED DESCRIPTION OF THE INVENTION

In the preferred embodiment of the present invention, we provide ten bioactive composites with different compositions, named a first composite, a second composite, . . . to a tenth composite, and the compositions of the composites are listed in the Table 1.

TABLE 1

| | | | | | PEEK |
|---|---|---|---|---|---|
| composite | CaSO₄ (%) | Ta₂O₅ (%) | BaSO₄ (%) | Fe₃O₄ (%) | (%) |
| 1 | 10 | | | | 90 |
| 2 | 10 | 10 | | | 80 |
| 3 | 20 | | | | 80 |
| 4 | 20 | 10 | | | 70 |
| 5 | 30 | | | | 70 |
| 6 | 30 | 10 | | | 60 |
| 7 | 40 | | | | 60 |
| 8 | 20 | 20 | | | 60 |
| 9 | 10 | | 10 | | 80 |
| 10 | 10 | | | 10 | 80 |

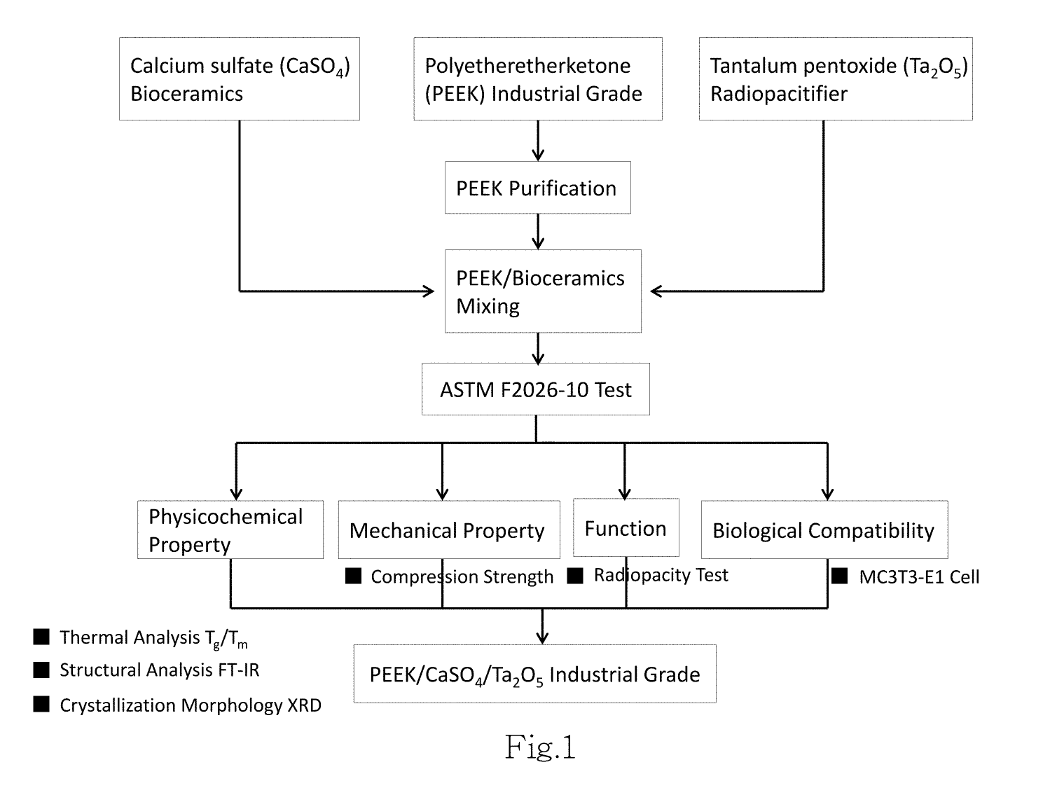

FIG. 1 shows a flowchart of manufacturing and testing processes of the bioactive composite of the preferred embodiment of the present invention. All the ten composites as described above have polyetheretherketone (PEEK). In the manufacture process, technical grade PEEK is selected and purified, and then is mixed with calcium sulfate (CaSO₄) bioceramics and tantalum pentoxide (Ta₂O₅), which is a radiopacity material. Next, we take some tests to test the composites whether they are the materials for surgery implants, which includes 1) functional test (for radiopacity); 2) physicochemical property test (thermal analysis (T_{g}/Tₘ), FT-IR, and XRD); 3) mechanical property test (compression strength); and 4) ISO 10993-5 MC3T3-E1 cell test. The results of the tests 1), 2), and 3) are described hereafter.

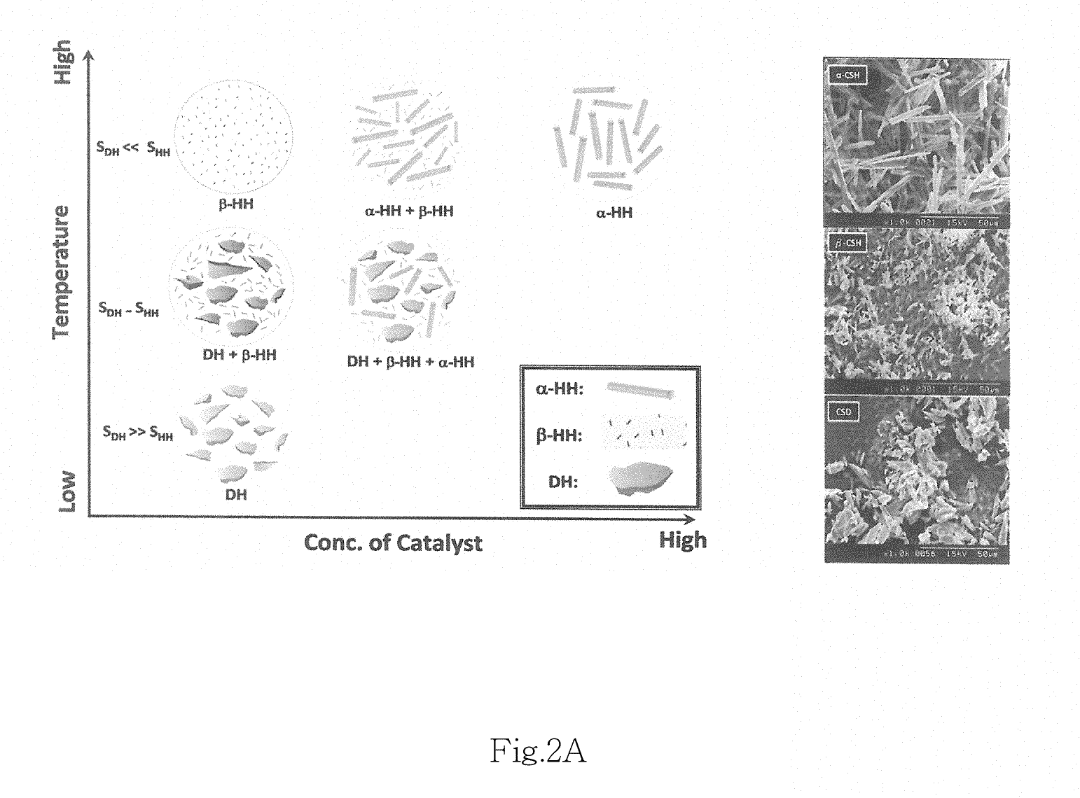

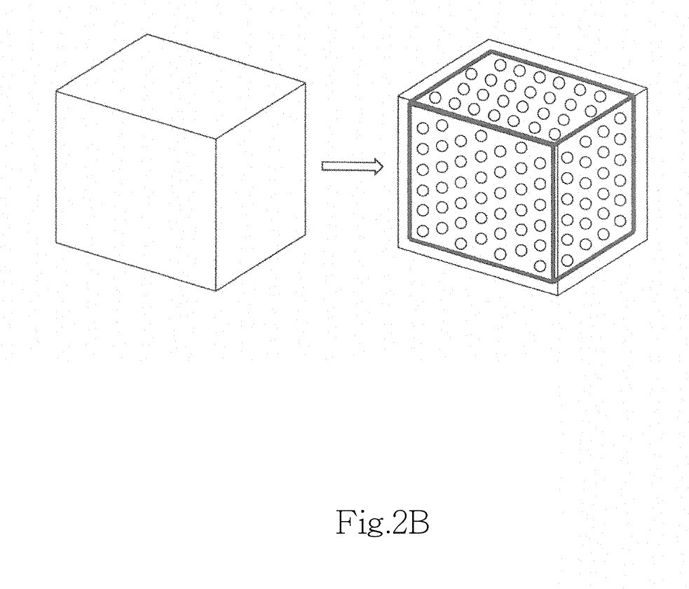

FIG. 2A shows various phases of calcium sulfate (CaSO₄) and manufacture conditions, and FIG. 2B shows that bioactive is increasing when calcium sulfate (CaSO₄), which has high solubility, is implanted in human body in the beginning, and then calcium sulfate (CaSO₄) will be dissolved to form a porous structure, which form porous scaffolds. The chemical reaction is:

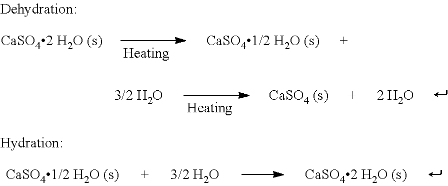

As shown in FIG. 2A, calcium sulfate (CaSO₄) has calcium sulfate dehydrate, alpha calcium sulfate hemihydrate, beta calcium sulfate hemihydrate, and anhydrous calcium sulfate. Moisture content of calcium sulfate will be volatized in the manufacture process, which causes the composites degrading. Therefore, smaller size benefits the process, and gets more uniform composite. In the present embodiment, the crystallization water in the beta calcium sulfate hemihydrate is removed to get anhydrous calcium sulfate. The anhydrous calcium sulfate is selected to be mixed with PEEK.

Furthermore, since calcium sulfate is hydrolysable, it could hydrolyze to form the composite of the present invention automatically when put it in human body. Besides, it will form pores when it is put in human body for a long time (FIG. 2B). When the composite of the present invention is applied to be implants inserted into intervertebral of human's spine, the implants must have sufficient strength to take the stress of a wide range of motion of spine. Calcium sulfate makes the composite of the present invention have such property.

In order to apply the composite of the present invention to be surgical implants, the composite satisfies Standard Specification for Polyetheretherketone (PEEK) Polymers for Surgical Implant Applications of American Society for Testing and Materials (ASTM F2026-10), and the requirements are listed in Table 2.

TABLE 2

| Parameters | Method | Requirement |
|---|---|---|
| T_{g} (° C.) | DSC, 20° C./min, sealed sample, | 125-165 |
| | T_{g} taken from second reheat | |
| Tₘ (° C.) | DSC, 20° C./min, sealed sample, | 320-360 |
| | Tₘ taken as max. point on reheat | |
| | exotherm | |
| T_{c} (° C.) | DSC, 20° C./min, sealed sample, | 260-320 |
| | T_{c} taken as max. point on | |
| | cooling endotherm | |
| Viscosity | Per 5.3 as agreed | As agreed |
| Infrared spectrometer | | per 5.1 |
| Total heavy metal as | U.S. Pharmacopeia Test 231 | <0.1 |
| lead. Max. % | | |

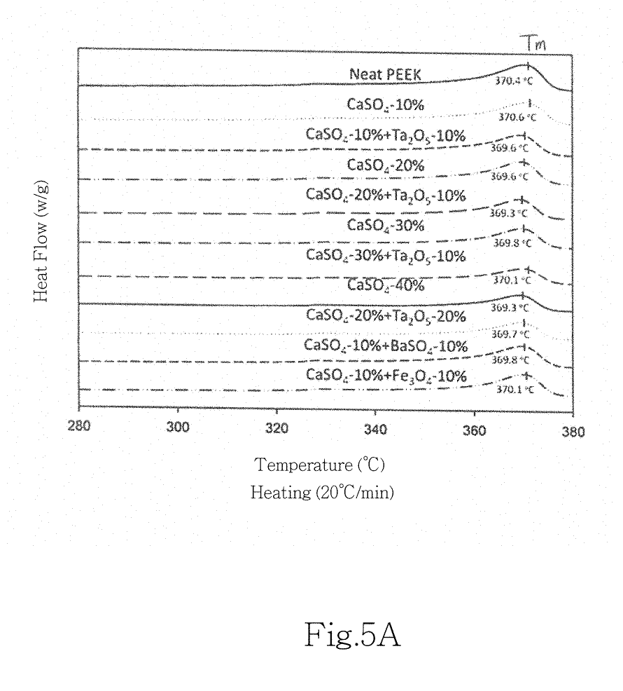

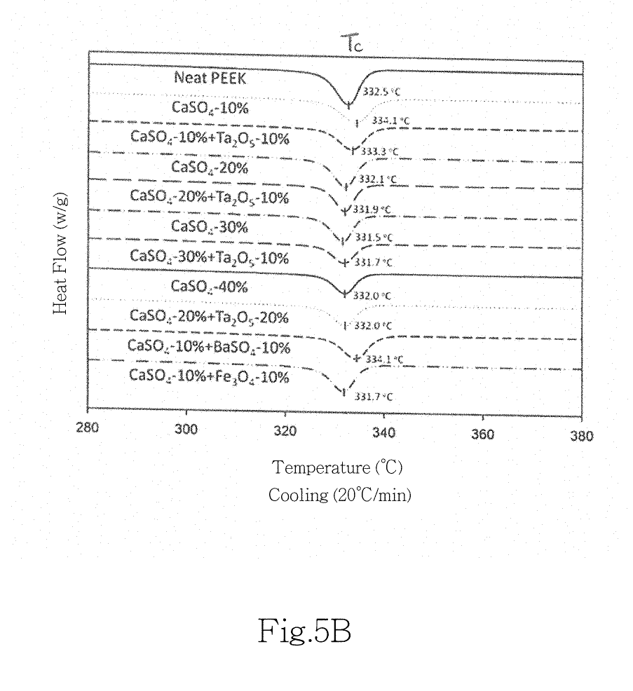

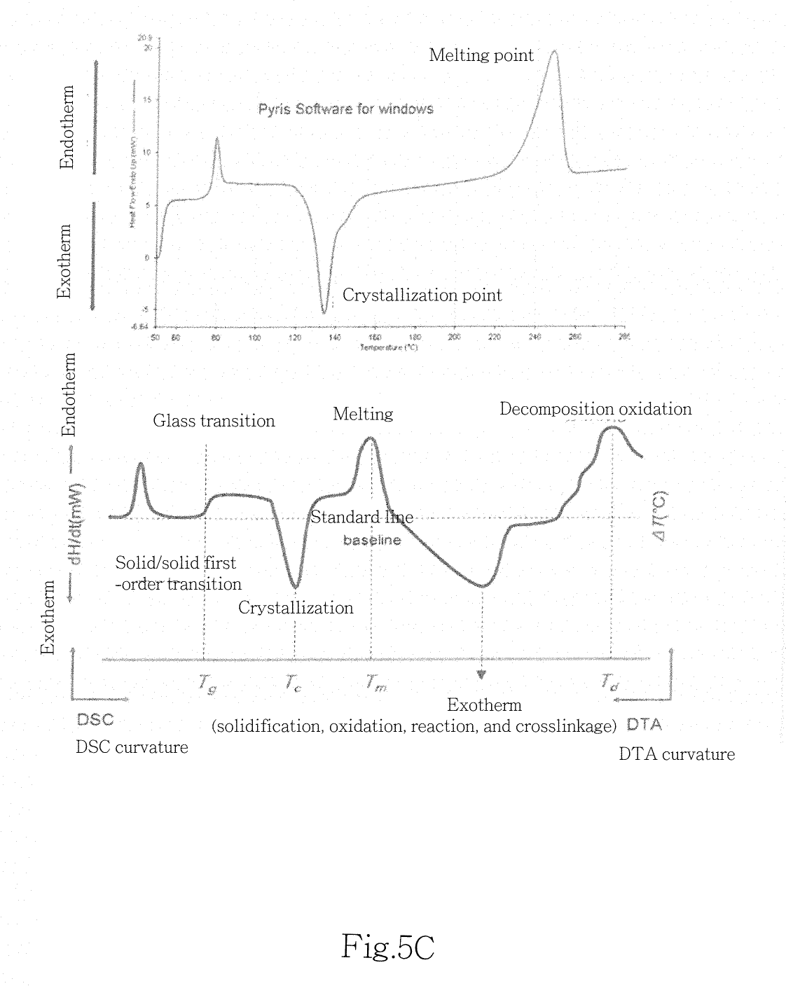

FIG. 5A and FIG. 5B show the melting temperature (Tₘ) and crystallization temperatures (T_{c}) of the ten composites provided by the present preferred embodiment, which are test by a differential scanning calorimetry (DSC). The principle of DSC is to measure the thermal energy to keep the temperature of the sample constant when the sample absorb or release heat due to phase transition, glass transition, and chemical reaction.

Typically, the reactions of polymer are shown in following table.

| | |
|---|---|
| No phase | Keep the temperature of the sample the same as reference by |
| transition and | overcome the difference of specific heat between them to show the |
| other reaction | standard line of DSC. In order to make sure the consistence of the |
| | standard line, the reference must have no chemical reaction in the |
| | experience temperature range, and have a constant specific heat. |
| Glass transition | When polymer approaches the glass transition temperature, heat |
| | capacity increases, it needs more heat to maintain the temperature, |
| | so the standard line of DSC usually changes. |
| Crystallization | Amorphous polymer, which is formed in cooling process, will |
| | crystalize when heating, and release crystallization heat. It must |
| | reduce the heat flow to keep the temperature constant, so the DSC |
| | has a heat release peak. |
| Melting | As the temperature raising, the crystalized part starts to melt, the |
| | compensator found that it has to increase the heat flow to keep the |
| | temperature, so a heat absorption peak generates. |
| Oxidation and | Some polymer has oxidation and crosslinking in high temperature, |
| crosslinking | so DSC line will have a heat release peak. |
| Decomposition | When the temperature is higher than a predetermined temperature, |
| | polymer chains break down, and a heat absorption peak appears. |

It is noted that glass transition temperature (T_{g}) is one of transition temperatures. Polymer will be transformed from a rubber type in high temperature into a glass type (hard and brittle) in low temperature.

FIG. 5C is the typical DSC thermograms showing the characteristic melting temperature (Tₘ) and glass transition temperature (T_{g}). Melting temperature (Tₘ), usually associating with processing temperature, is the temperature of the crystalline part of polymer breaking down by heat. Crystalline plastic has a significant melting temperature. In solid type, it has a regular molecular arrangement, higher strength and tensile stress, specific volume change a lot when it is molten, large shrinkage when it is solidified, internal stress can't be released easily, product is opaque, low heat dissipation, large shrinkage in cooling molding, and small shrinkage in hot molding. Amorphous plastic has no significant melting temperature. In solid type, it has an irregular molecular arrangement, little change of specific volume when it is molten, little shrinkage when it is solidified, product is transparent, color change into yellow when temperature is high, and has high heat dissipation.

As the discussion above, the ten composites of the preferred embodiment of the present invention includes the following specifications: crystallization temperatures (T_{c}) is between 330° C. and 333° C.; glass transition temperature (T_{g}) is 152° C.; and melting temperature (Tₘ) is between 368° C. and 370° C. The results show that all the composites of the preferred embodiment of the present invention satisfy ASTM F2026-10.

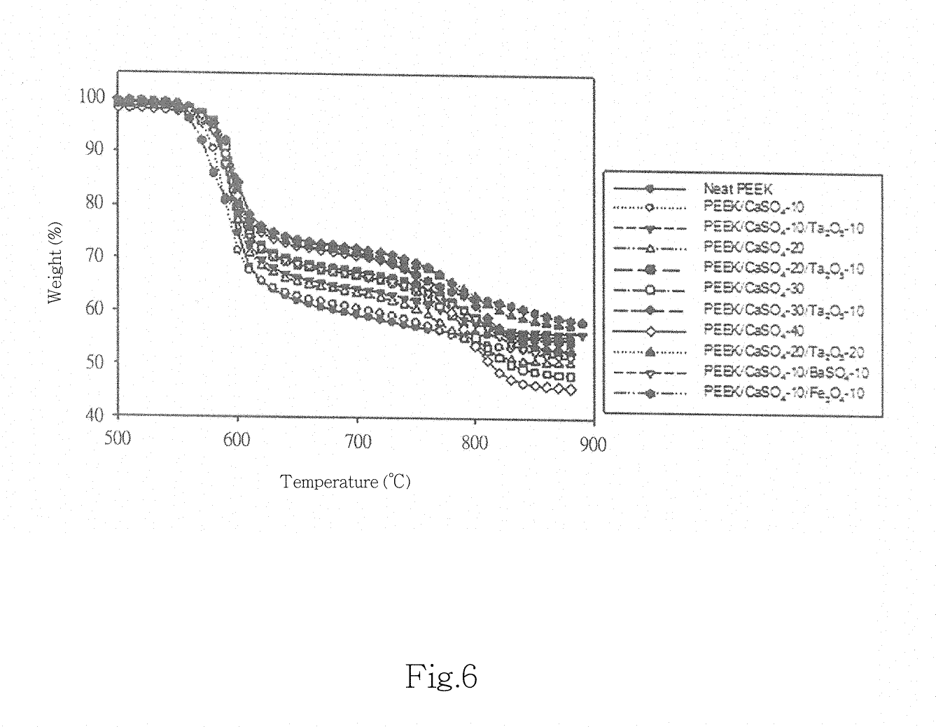

FIG. 6 shows the results of thermogravimetric analysis (TGA) of the ten composites of the preferred embodiment of the present invention. TGA measures a weight difference of a sample in a predetermined temperature. A sample is placed on a temperature-controllable heater. A predetermined gas, such as nitrogen and oxygen, is provided. The weight loss of the sample is recorded by temperature or by time to determine some properties of the sample, including degradation temperature, thermal stability, compositions, purity, moisture, reduction temperature, and anti-oxidation.

The minimum weight that TGA could measure is 0.1 μg, so the carriers should be received in a sealed chamber to prevent interference of air ventilation. The gas flow is limited for not to interfere the system.

TGA may set different temperatures. It could solve the problem of a sample having different decomposition rates, and making the products of decomposition overlap. In TGA, we may provide a constant temperature for the first stage reaction, and then raising the temperature to another constant temperature for the second stage reaction when the first stage reaction is totally completed that could obtain precise weight loss data.

FIG. 6 shows the results of TGA of the ten composites of the preferred embodiment of the present invention. It shows that a temperature range is between 570° C. and 590° C. for 10% weight loss.

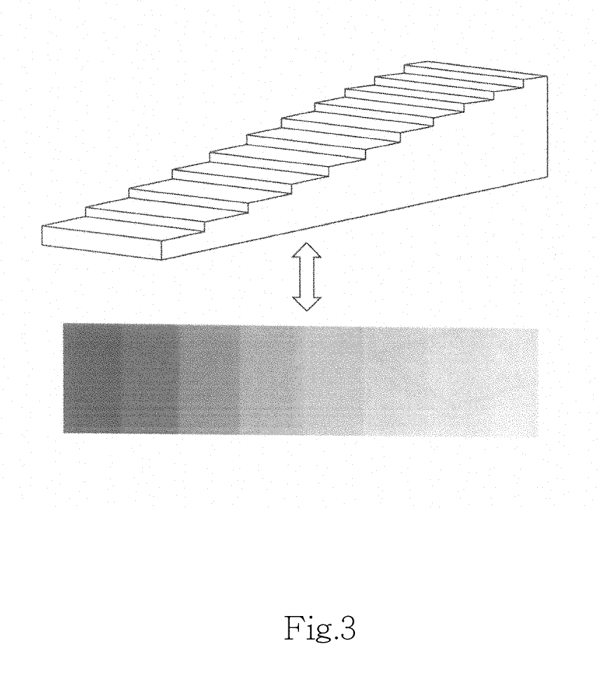

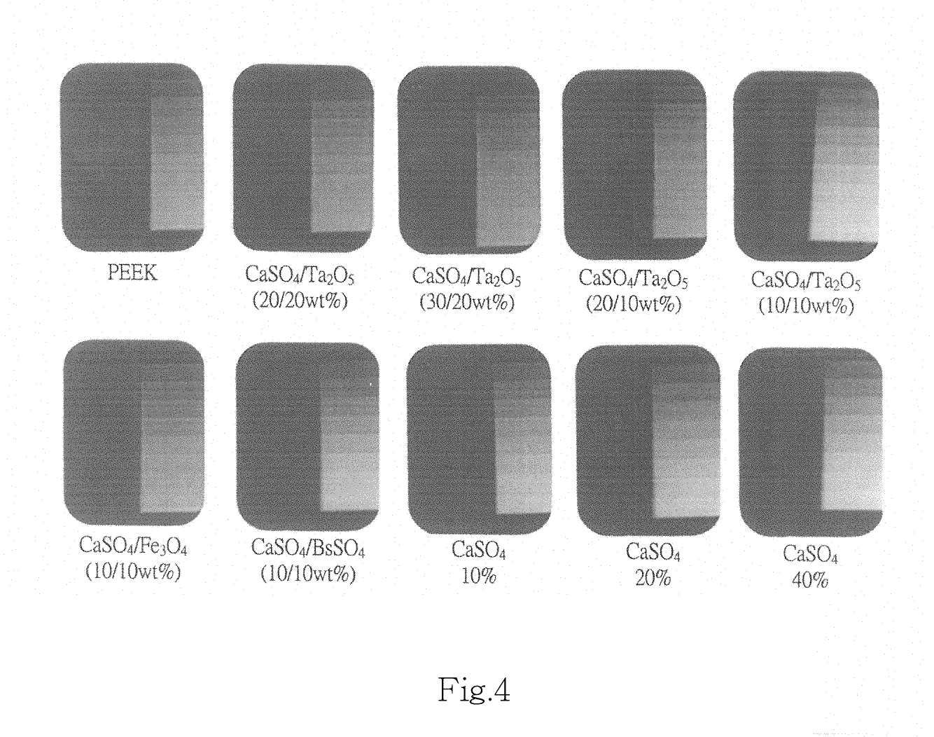

FIG. 3 is the reference gray scale relating to the thicknesses of aluminum corresponding to the X-ray radiopacity, and FIG. 4 is the gray scale of X-ray radiopacity of the ten composites of the preferred embodiment of the present invention. It shows that the radiopacity of the composites with tantalum pentoxide (Ta₂O₅) is better than that without tantalum pentoxide (Ta₂O₅), and the content of tantalum pentoxide (Ta₂O₅) is positive proportional to the radiopacity. The data are shown in Table 4. FIG. 4 also shows the ninth composite and the tenth composite having the most suitable radiopacity. Therefore, tantalum pentoxide (Ta₂O₅), ferrous ferric oxide (Fe₃O₄), and barium sulfate (BaSO₄) are the best additives of PEEK.

TABLE 4

| Group (N = 3) | Al step-wedge (mm) |
|---|---|
| CaSO₄ 20% | 0.00 |
| CaSO₄ 20% + Ta₂O₅ 20% | 1.22 ± 0.04 |
| CaSO₄ 30% + Ta₂O₅ 10% | 1.14 ± 0.05 |
| CaSO₄ 20% + Ta₂O₅ 10% | 1.18 ± 0.01 |

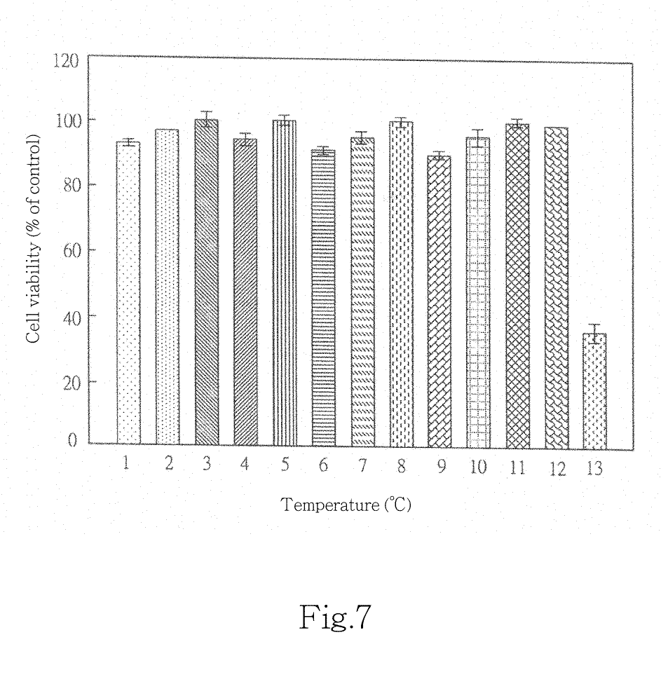

FIG. 7 is the biological compatibility of the ten composites of the preferred embodiment of the present invention and three control groups. The control groups include neat PEEK, positive control, and negative control. The data are shown in Table 5.

TABLE 5

| composite | CaSO₄ | Ta₂O₅ | BaSO₄ | Fe₃O₄ | PEEK |
|---|---|---|---|---|---|
| 1 | 10% | | | | 90% |
| 2 | 10% | 10% | | | 80% |
| 3 | 20% | | | | 80% |
| 4 | 20% | 10% | | | 70% |
| 5 | 30% | | | | 70% |
| 6 | 30% | 10% | | | 60% |
| 7 | 40% | | | | 60% |
| 8 | 20% | 20% | | | 60% |
| 9 | 10% | | 10% | | 80% |
| 10 | 10% | | | 10% | 80% |

| | |
|---|---|
| 11 | Neat PEEK |
| 12 | Positive control |
| 13 | Negative control |

Positive control: HDPE film extracted with 0.1 g/l ml MEM solution

Negative control: (0.5% DMSO)

All the composites pass MC3T3-E1 based cell viability test.

It must be pointed out that the embodiments described above are only some preferred embodiments of the present invention. All equivalent structures which employ the concepts disclosed in this specification and the appended claims should fall within the scope of the present invention.

What is claimed is:

1. A bioactive composite comprising: 10% to 40% by weight of calcium sulfate (CaSO₄), 10% to 20% by weight of tantalum pentoxide (Ta₂O₅), and 40% to 80% of polyetheretherketone (PEEK);
wherein calcium sulfate is anhydrous calcium made by removing crystallization water of beta calcium sulfate hemihydrate.

2. The bioactive composite of claim 1, further comprising up to 10% by weight of barium sulfate (BaSO₄).

3. The bioactive composite of claim 1, further comprising up to 10% by weight of ferrous ferric oxide (Fe₃O₄).
